(19) United States
(12) Patent Application Publication
(10) Pub. No.: US 2010/0324544 A1
(43) Pub. Date: Dec. 23, 2010

(54) OPTICAL TREATMENT SYSTEM AND AN ADJUSTMENT MEMBER THEREFOR

(75) Inventors: Rembert Fertner, Klagenfurt (AT); Guido Roosen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/446,260

(22) PCT Filed: Oct. 16, 2007
(86) PCT No.: PCT/IB07/54212

§ 371 Date: Apr. 20, 2009

(30) Foreign Application Priority Data
Oct. 23, 2006 (EP) 06122755.9

(51) Int. Cl. A61B 18/18

(52) U.S. Cl. 606/9

(57) ABSTRACT

The invention provides an optical treatment system comprising a first optical window (20), a light source (14), a control unit (18) for controlling the light source (14) such that optical radiation at the first optical window (20) has at least one optical parameter with a predetermined first value, and comprising at least one transmitting adjustment member (30) having a second optical window (34) and being positionable such that optical radiation is emittable via the second optical window (34), said optical treatment system further comprising an optical parameter control means (36, 38; 42; 46, 48, 50; 52; 54) that is arranged to control said at least one optical parameter to have a predetermined second value at the second optical window (34). The optical parameter control means may be active, and influence the control unit (18) or light source (14), and/or passive, and influence the radiation emitted through the first optical window (20) by filtering, reflection, absorption, etc. Thus, the system is able to provide radiation having well known, predetermined and thus more reliable properties, even if the second window has a different shape or area or the radiation has been influenced otherwise.

### FIELD OF THE INVENTION

The invention relates to an optical treatment system according to the preamble of claim 1, and in particular comprising a housing with a first optical window, a light source in the housing for providing optical radiation, a control unit for controlling the light source, such that the system is able to provide optical radiation at the first optical window with at least one optical parameter having a predetermined first value, and a set of at least one optical radiation transmitting adjustment member with a second optical window, the adjustment member being positionable with respect to the light source such that at least a part of the optical radiation is emittable via the second optical window.

### BACKGROUND OF THE INVENTION

Document WO99/58195 discloses an apparatus for selective thermolysis of the skin, comprising inter alia a housing with a cavity and an opening, and a pulsable source of narrow band electromagnetic radiation to selectively heat target tissue within the skin. The apparatus preferably includes an extension with an aperture. Shape and area of the aperture may vary.

This known apparatus has the disadvantage that, because of the use of such an extender, reliable and safe use thereof may not be guaranteed under all circumstances. In particular, the known apparatus treats the skin by heating the tissue with radiation. To control the apparatus, skin temperature is measured. However, apart from skin temperature also other parameters play a role in skin safety, in particular when treating skin with radiation. By applying an extender to a treatment apparatus, such other parameters may change, and may for example exceed a safety threshold, causing damage or pain.

### SUMMARY OF THE INVENTION

It is an object of the invention to provide an optical treatment system of the kind mentioned above, that is more reliable when using different adjustment members.

According to the invention, this object is achieved with an optical treatment system according to the preamble of claim 1, wherein the system further comprises an optical parameter control means that is arranged to control said at least one optical parameter to have a predetermined second value at the second optical window. Herein, controlling said at least one optical parameter includes influencing and changing said optical parameter. By providing a control means of this kind, the system according to the invention is much better defined, in that the optical parameters are known and controlled to a predetermined value before use of the system, instead of using a feedback signal from treated skin relating to a single effect of the applied radiation. When an adjustment member is used, the consequences for the optical radiation at the level of the “new” second optical window are known beforehand, and unwanted side effects by applying optical radiation with one or more incorrect parameter values may be prevented. Examples of the latter are too little fluence or intensity, or a too high fluence or intensity, which may cause ineffectiveness or damage/pain, respectively, and also a wrong spectrum, which may cause either.

Contrarily, the known apparatus only controls the pulsed electromagnetic radiation by checking a skin temperature, i.e. by checking one of the possible consequences of its treatment. This check on temperature is insufficient and unreliable when performing other optical treatments. No reliable check on optical parameters is present when changing the shape and/or area of the optical window by applying different extenders.

In the context of the present invention, the light source need not be present as a complete unit within the housing. Alternatively, an external light generating means could be provided, having one or more lightguides to guide the generated light to the housing, with the accompanying advantages of smaller weight, more flexibility etc. The end(s) of the lightguide(s) could then be considered as the light sources. Similarly, the control unit may also lie outside the housing, and may e.g. be provided close to the light source. The control unit may also be provided distributedly, i.e. a part with circuitry outside the housing, and a part such as sensors or detectors within the housing. In use, the distributed parts will co-operate and/or communicate with each other. The above possibilities are deemed comprised in the invention.

Moreover, the optical window may either be an opening in the housing or the member, or a physical entity such as a transparent element, for example a filter or a glass plate. Furthermore, the first and/or second optical window may be present in such a position that, when the system is in use, the first and/or second optical window are pressed against the skin to be treated. However, it is also possible that the actual optical window is kept away from the skin, such as by a kind of collar. A corresponding correction of parameter values is deemed to be both comprised within the present invention and within reach of the skilled person. It is noted that the second value relates to the optical parameter's value immediately after said second optical window, and not just before or inside the second window. If the second optical window is an optical element, the second value could be determined at the outer surface thereof. In case the second optical window is an aperture, such as in the presence of a collar, the second value could be determined at the level of the skin contacting the adjustment member. Other definitions are not excluded however, and it is only relevant that the second value is known beforehand, such that e.g. no feedback from the skin during treatment is required to set the light source.

Furthermore, the system may comprise other means to be able to provide the required radiation, such as filters. The skilled person may easily select such other means to obtain the desired properties of the radiation, and these means are deemed also comprised in the present system. In this respect, ‘optical radiation’ is intended to include visible radiation as well as ultraviolet radiation in the UV-A and UV-B regions, preferably only the UV-A region, as well as near infrared region. Expressed in wavelengths, optical radiation is meant to span the range from about 300 nm to about 2000 nm, preferably from about 400 nm to about 1500 nm, most preferably from about 400 nm to about 1000 nm.

Further advantageous embodiments are disclosed in the dependent claims.

In particular, the adjustment member may be a cap, with a continuous sidewall and an internal cavity, which may be covered on one or two sides with a transparent covering. Other embodiments, such as a solid transparent body, are not excluded.

In an embodiment, the optical parameter control means is arranged to control said optical parameter in dependence on at least one of a shape and a dimension of said second optical window and/or adjustment member. The shape of the optical window may relate to a certain treatment zone, such as a face, the bikini area or an armpit, with possibly special requirements as to the optical parameter, such as fluence. The adjustment members as used in the present invention serve to provide a system that is better suited to treat certain areas of the skin. In some cases, the skin is more sensitive, and lower values of certain parameters should be used. A correlation between a specific adjustment member for such areas and the original (main) system without the adjustment member may be made. In other cases, an area is too curved or too small to be treated with the standard (first) optical window, and the adjustment member is required to reach those areas. Other or the same values of the optical parameters may apply. For example, the treatment may relate to shaving or epilating by means of optical radiation, or treatment of port-wine stains, etc. The dimension, i.e. mostly the area, of the second optical window, and possibly also the shape of the adjustment member, influence the radiation that is actually emitted through the second optical window. The invention provides an adjustment means to correct this influence, and to bring the optical parameter to, or maintain it at, a desired value.

In a special embodiment, the optical parameter comprises at least one of a spectrum, an intensity and a fluence of the optical radiation, and preferably the intensity. The adjustment member may influence the radiation by absorbing or reflecting a wavelength-dependent part thereof. In other words, the spectrum may change, and it is of course worthwhile to control the spectrum to correct such a change. Alternatively or additionally, a different shape or dimension of the adjustment member may indicate a treatment area with special requirements as to the spectrum, e.g. a thicker skin, a more sensitive skin or a suntanned skin, which may need a different spectrum, such as less infrared radiation in the case of a more sensitive skin. Moreover, a particular hair colour in the case of optical shaving may also require a different spectrum.

Providing a desired spectrum may be done by supplying optical radiation at the first optical window or at least at the light source (primary radiation) with a complementing, correcting spectrum. Advantageously, the optical parameter control means comprises at least one optical element that has a wavelength-dependent absorption and/or reflection. Such an optical element could be a filter or selective mirror or the like. Also possible is a light source with a tunable spectrum.

In many cases, intensity and fluence are both important parameters, and controlling at least one of them often prevents harmful effects or ineffectiveness. Generally, this relates to the average intensity or fluence across the optical window. Note that intensity and fluence are related through the time during which radiation is supplied, and they will vary similarly if that time is not changed.

In an advantageous embodiment, the second value is substantially equal to the first value, and in particular deviates no more than 20%, more particularly no more than 10%, from the first value. Assuming that the system is designed such that the functionality, safety and so on are satisfactory, or even optimal, for radiation delivered at the level of the first optical window, it will be advantageous for radiation with an equal value at the level of the second optical window.

Alternatively or additionally, at least for one parameter the second value is higher if a dimension, in particular the area, of the second optical window is smaller than a corresponding dimension, in particular the area, of the first optical window, and preferably by a factor that corresponds to the ratio of the skin scattering loss factor for the second optical window and the skin scattering loss factor for the first optical window. Preferably, said loss factor is dependent on the desired treatment depth. This takes into account that, when the optical window becomes smaller, the optical properties of the skin, and in particular scattering, becomes more important. For a large first or second optical window, for example with a diameter of about 20 mm, scattering is not particularly relevant for suitable radiation. On the other hand, if e.g. the second optical window is much smaller, for example with a diameter of about 3-5 mm, this scattering becomes important, especially for treatment depths of at least a few tenths of a millimeter. Then there will be a relatively increased scattering loss by a certain factor as can be easily determined in practice for the specific case. By correcting for this factor, fluence values may be controlled, such as maintained at a constant value.

In another embodiment, the second value is lower than the first value. This relates in particular to adjustment members for more sensitive areas. Note that it could easily happen that because of the smaller dimensions and due to internal reflection, the second value would be higher, which is undesirable in that case. The invention prevents this from happening with suitable optical parameter control means. Note that it is also possible to adjust the light source, but in some cases that could be undesirable.

In a special embodiment, the adjustment member is removably attachable to the housing, around the first optical window. This is a very suitable embodiment, in which one or more adjustment members are provided which may be operably attached to the housing. To this end, attachment means, such as a thread, magnets, a simple collar and so on, may be provided to the housing, the adjustment member or both. A user simply attaches, removes or exchanges a suitable adjustment member.

In another embodiment, the adjustment member is attachable or attached to the housing, with the housing being adjustable with respect to the light source, such that the second optical window is positionable in front of the first optical window, or such that the first and second optical windows switch positions. In this embodiment, the permanently or removably attached adjustment member is not always present in an operable position, such as before the first window, but may be adjustable, such as rotatable or shiftable, towards such an operable position. For example, the adjustment member is attached, in a first position, on a rotatable etc. brace or bracket or the like. Then, the adjustment member is rotated, shifted etc. into the operable second position. It is also possible to provide a housing that has two, or more, optical windows, said housing being adjustable, e.g. rotatable or shiftable, such that a selected optical window may be operably positioned with respect to the light source.

In a particular embodiment, the optical parameter control means is arranged to adjust the control unit. In other words, the optical parameter control means is an active control means that is able to initiate and/or control a setting of a part of the system outside the adjustment member, such that a desired, predetermined value of an optical parameter is obtained. In this way, there is a good reliability of the system, because of the active control of its settings via control by the adjustment member. Such adjusting of settings may e.g. relate to the light source power, the energy per flash and so on. Examples of how this control may be achieved will be given below, although embodying this feature is already deemed within reach of the skilled person.

In an embodiment, by means of a pulsed light source, the optical parameter control means provide instructions to, or control directly, a power source for the light source, such as a discharge unit, comprising e.g. a capacitor. A simple way of selecting a power level is to select a lowest voltage for the capacitor that is reached after discharge. Other ways are easily implemented by the skilled person, and may e.g. be given electronically, as a set of instructions to power source or control unit.

In a particular embodiment, an area of the second optical window is larger than an area of the first window. This embodiment uses the circumstance that the active control allows e.g. a higher energy per flash or time period of the radiation, which is required when this energy has to be applied to a larger area with e.g. the same fluence. Without active control, attaching an adjustment member to a second optical window with a larger area would not result in the same fluence. Having a larger optical window may be advantageous, e.g. to be able to treat larger areas in one go, such as legs or the back, without having to provide a large-area first optical window, which would make the system as a whole too bulky for easy handling during treatment of smaller areas.

In a special embodiment, the optical parameter control means comprises circuitry that is arranged to communicate with the control unit. This circuitry may be arranged, such as programmed, to instruct the control unit, or to control the light source directly. For example, such circuitry may set a treatment duration, such as a pulse length or pulse number, to set a desired predetermined fluence. It may also set the light source, such as the power or spectrum thereof, or it may trigger the operation of an additional means, such as insertion of a filter or mirror, in order to influence and control the optical radiation. The term circuitry should be construed in a broad sense, and comprises so called ICs, and other known electronic and electrical devices by which control may be exerted, such as by providing a code.

In a special embodiment, the optical parameter control means comprises an identification means that is able to co-operate with the control unit. Such an identification means serves to provide the required information to the control unit to adjust settings for the light source. In this way, as well as with the above mentioned circuitry, which is also an embodiment of the identification means, it is very easy to provide each adjustment member with clear and unambiguous means to set a correct radiation parameter. Such an identification means need not communicate with a control unit or light source. It is enough when the identification information may be extracted in a suitable way, such as when it is readable by the control unit. Note in this respect that a separate identifier means may be provided on the housing, but since this should co-operate with the control unit, such a separate identifier means is deemed an at least functional part of the control unit. Possible examples of identification means (and identifier means) are a bar code (and a barcode reader), the passive (readable) circuitry, such as RF-ID ICs, or mechanical means, such as switches or spring loaded pin means. Even with mechanical means it is possible to identify various adjustment members, e.g. by providing multiple switches that are operable by adjustment members having different means for operating the switches. In a particular embodiment, the housing comprises a detector means arranged to detect the presence of an adjustment member.

In an embodiment, the detector means is arranged to detect the type of the adjustment member. Such detector means may be embodied as described above, i.e. in the form of one or more switches et cetera. By providing such detector means, again, simple detection of whether or not an adjustment member is installed may be made, and possibly one or more settings of or for the light source may be adjusted.

In another embodiment, the optical parameter control means comprise a passive radiation influencing means, that is arranged to manipulate the optical radiation emitted by the light source. In particular, the optical parameter control means are arranged to manipulate the optical radiation emitted through the first optical window. In both cases described here, the manipulation is such that the desired value for the optical parameter is obtained at the level of the second optical window.

Each adjustment member will have some influence on the optical radiation as emitted by the first optical window, or at least as emitted by the light source if the first and second optical windows are switched. Some radiation could be absorbed, some radiation could be reflected, et cetera. These changes are caused by the various surfaces and materials of which the adjustment member is composed. Most generally, the passive radiation-influencing means comprise the whole of these surfaces and materials, provided that the net effect is that the optical parameters of the radiation at the level of the second optical window are as desired, i.e. are of the predetermined value(s). This is extremely unlikely, however, since it is possible only by chance. The distinguishing feature is still, however, that the radiation at the second optical window does have that (those) predetermined parameter value(s), which makes the radiation known and of a predetermined quality. It is of course also possible to provide one or more additional passive radiation-influencing means, or means such as materials that have been especially selected, in order to obtain just the desired value(s) for the optical parameter(s). From now on, the term ‘parameter’ is meant to include the plural as well.

More in particular, said value is equal for the first and second optical window. This is a very advantageous embodiment, as already described above, in that special care has been taken in the adjustment member to ensure this constant value, by providing additional or at least suitable radiation influencing means. Such means may be embodied in very many ways, as long as it is ensured that the optical parameter has the desired predetermined value at the second optical window. Some special radiation influencing means will be described below.

First, it is remarked that in most cases the adjustment member comprises a hollow space or cavity. Then, the inner wall of the adjustment member should be at least partly reflecting, i.e. sufficiently reflecting to compensate for losses or the increase in radiation intensity. A suitable reflection may be determined in practice, e.g. by calibrating or gauging, or may be calculated on the basis of a physical model, etc. In all of the embodiments described below, it is assumed that such calibration or calculation has taken place, which is within reach of the skilled person.

The adjustment member could be provided with a rim or edge comprising a different material. Especially if the adjustment member is used on particular or sensitive parts of the skin, selecting an appropriate material may be advantageous. For example, for the skin in the armpits or at the bikini line, a supple rubber would be advantageous. In addition or alternatively, the skin contacting parts of the adjustment member may be specially shaped. In particular, and for similar reasons, the skin contacting edge of the adjustment member could be made more rounded.

In a particular embodiment, an inner wall of the adjustment member is at least partly reflecting, in particular specularly reflecting, and has an inner diameter that decreases in a direction towards the second optical window. In this embodiment, the radiation from the first optical window, that is mostly emitted in a large range of angles, will be reflected by the inner wall. Since an inner diameter decreases, opposite parts of the inner wall will be angled towards each other. This ensures that a part of the radiation, after one or more reflections, will travel back towards the first optical window (or light source), or at least is removed from the radiation towards the second optical window. This compensates the fact that in principle all of the radiation emitted by the first optical window would travel towards and through the second optical window, apart from absorption losses etc. The skilled person may select a suitable (e.g. apex) angle of the wall of the adjustment member, a suitable reflection coefficient, a suitable type of reflection, i.e. diffuse or specular or a mixture, a suitable part of the surface area of the inner wall et cetera, to obtain the desired parameter value.

In a special embodiment, an inner wall of the adjustment member is partly absorbing, e.g. sufficiently absorbing to compensate for the other losses and/or for an increase in radiation intensity as described above. To this end, the inner wall and other parts of the adjustment member could be coated with a coating having a suitable absorption coefficient, and having a suitable surface area, to obtain the desired predetermined optical parameter value. Again, the skilled person can easily obtain such a coating or the like, by calibrating, trial and error, calculation and so on.

In a special embodiment, the adjustment member comprises at least one optical element. Herein, ‘optical element’ means any light transmitting part in the adjustment member, such as present as the second optical window, or in addition thereto. Note that it may suffice for the correction to have a suitable optical element as the second optical window, since any element will have a reflectance. For example, normal glass reflects about 4% at each glass air interface, which reflection may be sufficient to obtain the desired optical parameter value. Of course, it is possible to provide more than one optical element, e.g. to increase the total reflection loss.

In an embodiment, the optical element comprises a filter. This means that the optical element has a non-negligible absorption coefficient for at least part of the optical radiation. For this purpose, the filter could comprise a suitable material, such as coloured glass, or a neutral grey filter etc. Note that the absorption, and likewise the reflectance as discussed above, may be wavelength dependent, as may be required to obtain the predetermined parameter value (including spectrum).

In an embodiment, the optical element comprises a carrier with a coating that is partly reflective and/or absorptive. A big advantage of this embodiment is that after e.g. through calibration the radiation emitted at the second optical window without the presence of the radiation-influencing means in the form of this optical element with a coating on a carrier, may easily be exactly compensated, by applying a corresponding coating. The coating could be e.g. a metal layer, patterned or non-patterned, having a suitable thickness. The carrier could be e.g. a separate body, or could be the second optical window, if embodied as an optical element.

In another embodiment, the optical element comprises at least one lens, preferably with a focal length that is shorter than a length of the adjustment member, more preferably comprising a plurality of lenses in a condenser arrangement. By thus influencing the optical path of the radiation, the part that is directed towards an inner wall, that is e.g. absorptive or reflecting, may be increased, or at least a part of the radiation may be taken out of the ‘normal’ bundle towards the second optical window. In this way, the optical parameter value may be influenced. The embodiment with the condensor arrangement has the advantage that beam properties, in particular a range of angles, may be better controlled, such as for example made similar to the beam properties at the first optical window.

In a special embodiment, the adjustment member comprises, or even consists of, at least one lightguide, preferably a bundle of optical fibers, with a diameter that decreases towards the second optical window. In order to compensate for losses when coupling radiation into the lightguide, it will usually be necessary to concentrate the light by decreasing the diameter of the lightguide. This embodiment could be useful to ensure more flexibility. A lightguide may be made from optical fibers, and thus allow e.g. a small second optical window, for treatment of small or difficult to reach areas.

In a special embodiment, the optical radiation-influencing means comprise a combination of the above mentioned features. For example, reflecting inner walls could be combined with a filter at or near the second optical window. This ensures that most of the radiation is available at the level of the second optical window, and may be selected as desired by mounting a suitable filter. In order to obtain a high degree of reflection, the inner wall could be covered with a metal layer, or a diffusely reflecting material such as TiO2 etc.

In a particular embodiment, the system, and in particular the adjustment member, comprises cooling means arranged to remove heat from the adjustment member. In many embodiments, such as those in which the second optical window has a smaller surface area than the first optical window, and the parameter value is substantially equal, less light leaves the adjustment member than is emitted through the first optical window. The difference represents an amount of energy, which is often, though not always, absorbed by the system, and mostly by the adjustment member itself To prevent unwanted heating up of the adjustment member, it will be advantageous to remove this heat. Although some heat will be removed by radiation, convection cooling et cetera, without special measures, the provision of specially arranged cooling means offers advantages. Below, some special examples will be described, although the skilled person could also select other cooling means. Note that it is advantageous to provide a cooling means on the adjustment member, since there additional heat will often be absorbed, and it is often advantageous to control heat close to the source.

In an embodiment, the cooling means comprise a coolant fluid flow in thermal contact with the adjustment member. The coolant flow could be made controllable by a or the control unit, e.g. via one or more valves. The coolant could be drained away from the adjustment member, or could be carried to a part of the adjustment member, or the system in general, that is cooler or has a larger cooling surface, and so on. The coolant could be a liquid, such as water, or a gas, such as air.

In an embodiment, the cooling means comprise a coolant fluid channel in the adjustment member for flowing coolant fluid. Such a coolant fluid channel helps in directing the coolant to places that would heat up most, such as dark, absorbing parts. The coolant fluid channel could thus e.g. be guided along a part of the adjustment member, such as a wall or a filter.

Preferably, if the housing already comprises a cooling system, such as a coolant fluid channel, the cooling means of the adjustment member are connectable to the cooling system. Preferably, a connection between the cooling system of the housing and the cooling means of the adjustment member comprises at least a controllable inlet valve. This allows supply on demand of coolant fluid.

In a special embodiment, the cooling means comprise a coolant fluid channel in the adjustment member, in particular in a wall thereof. Often, a wall will absorb a large part of the radiation, and will thus heat up most, while a user could easily touch such a wall. To prevent hurt or even danger, it is preferable to cool the wall. This can efficiently be done by providing a coolant flow in the wall, preferably through a coolant fluid channel.

In another embodiment, the adjustment member comprises at least two optical elements, between which a coolant fluid channel is provided. If the adjustment member, or the system in general, comprises two or more optical elements, it will be possible and advantageous to provide a coolant fluid channel between those optical elements. Examples are a filter and a protection glass. The filter would heat up most, while the protection glass remains relatively cool, due to differences in absorption. A coolant flow between the filter and the protection glass could efficiently carry off heat from the filter.

In an embodiment, the cooling means comprises a coolant flow, in particular a gas flow in an inner space of the adjustment member, for example in the form of air conditioning, or cooling with some other cooling or cooled gas. An advantage could be that e.g. cooling air could be vented off into the atmosphere without objection.

In an advantageous embodiment, the system comprises a heat sink, such as a part with projections such as ribs. It preferably includes, in embodiments both with and without a heat sink, a coolant displacement means, such as a fan or a pump, to be able to provide fresh coolant to the heated up parts.

In an embodiment, a wall of the adjustment member comprises, and preferably is substantially composed of, a material with a thermal conductivity of at least 50 W/mK. Preferably, the wall comprises aluminium, or copper. This offers the possibility to guide heat quickly to other parts of the system, which in turn allows heat to be carried off by an already existing part of the system, such as a heat sink on the housing. But already the redistribution of heat could suffice to keep the temperature of the parts below desired limits, in cases where heat is generated quickly, but in small amounts, i.e. high-power, low-energy applications.

In a special embodiment, the cooling means comprise a coolant spray nozzle that is arranged to spray a coolant onto a part of the adjustment member. An example is a cryogenic spray, but a water spray etc. would also suffice. The latent heat of e.g. a phase transition provides a very effective cooling.

In a particular embodiment, the system provides radiation in a pulsed mode with a repetition frequency, and the cooling means are arranged to adjust the system such that the repetition frequency is lowered. E.g., the cooling means comprise an adjustment member-identification means to influence or control the control unit or light source. A similar principle as discussed above for controlling the light level may be applied here. Note, however, that it is not the power during irradiation or the total fluence which is adapted but rather the time period during which this fluence is supplied, in order to be better able to carry off the generated heat.

The invention also comprises an adjustment member for use in the optical treatment system according to the invention. A distinguishing feature is that this adjustment member comprises a means as described above that ensures that the optical parameter has a desired predetermined value at the second optical window, when used in a treatment system according to the invention. All features described above and relating to the adjustment member may distinguish this adjustment member according to the invention from known adjustment members. For brevity, these features are implied, but not explicitly repeated here.

### BRIEF DESCRIPTION OF THE DRAWINGS

The invention will now be further elucidated with reference to the drawing, in which a number of illustrative and non-limiting examples of an optical treatment system according to the invention are shown and described, and in which in particular:

FIG. 1 schematically shows a system according to the invention, in a side elevational view;

FIGS. 2a and b show another embodiment of the system, in a side elevational view;

FIGS. 3a-c show some embodiments of adjustment members 30, in a cross-sectional view;

FIG. 4 shows another adjustment member according to the invention, in a cross-sectional view;

FIG. 5 shows another embodiment of an adjustment member 30 in cross-sectional view;

FIG. 6 shows an adjustment member with cooling means, in a cross-sectional view;

FIG. 7 shows (a part of) a system with a number of different cooling means, in a cross-sectional view.

### DETAILED DESCRIPTION OF PREFERRED EMBODIMENTS

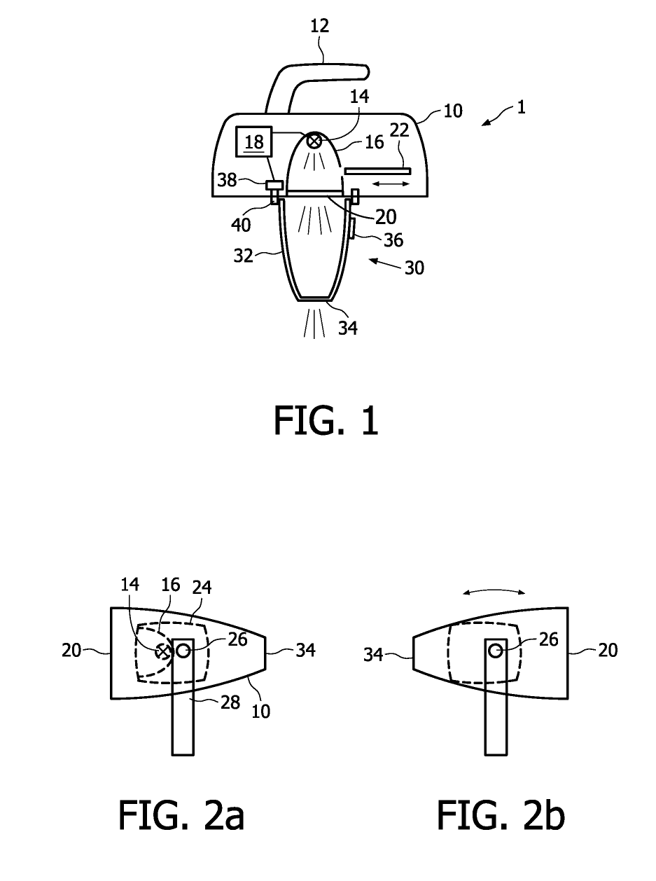

FIG. 1 schematically shows a system according to the invention, in a side elevational view.

The system comprises a main treatment device 1 and an adjustment member 30. The main treatment device 1 comprises a housing 10 with a grip 12. Within the housing 10, there is provided a light source 14 with a reflector 16, a control unit 18 and an optional filter 22. The housing also comprises a first optical window 20.

The adjustment member 30 comprises a wall 32, a second optical window 34 and optionally an ID-chip 36. The adjustment member 30 is attached to main device 1 by means of attachment means 40. Furthermore, 38 is a switch that is operable by attaching adjustment member 30.

The light source 14 may be any suitable light source, such as a flash lamp, a laser and so on. The light source 14 is controlled by control unit 18, in order to switch it on and off, to set an (average) power, possibly a spectrum, a pulse rate, and so on.

The radiation from the light source 14 is emitted via the first optical window 20, which is shown as an optical element, such as a glass plate, but which could also be an aperture. A filter 22, that is moveable in the direction of the arrows, under control of the control unit 18, could be inserted to influence the spectrum or power.

In FIG. 1, an adjustment member 30 has been attached to main device 1 by means of the attachment means 40, such as a collar, thread or snap-in connection. Attaching the adjustment member 30 brings the switch 38 into operation, which informs control unit 18 of the presence of the adjustment member 30. This adjustment member has a second optical window 34, which is smaller than the first optical window 20. The control unit 18 may thereupon change the setting of the light source 14, and/or insert filter 22, in order to ensure that an optical parameter of the radiation emitted at the second optical window 34 has a desired predetermined value, such as a constant fluence. Adjustment member 30 optionally has an ID chip 36 that may communicate with control unit 18 to exchange information about the adjustment member, the treatment etc.

FIGS. 2a and b show another embodiment of the system, in a side elevational view. Here, as in all of the drawings, similar parts are denoted by the same reference numerals.

In FIGS. 2a, 2b, the housing 10 itself comprises the first optical window 20 and second optical window 34. An internal part 24 comprises the light source 14 and a reflector 16, and is fixedly attached to a grip 28 in the shape of a brace or bracket. The housing 10 is rotatably attached to the grip 28 via a swivel axle 26.

A user can select the desired optical window 20, 34 by rotating the housing 10 around the swivel axle 26. A sensor or detector (not shown) is built into the system to detect which optical window is in front of the light source. This sensor or detector instructs the control unit to adapt the settings for the light source, where required. Alternatively, the optical windows may each comprise a filter, such that the desired optical parameter is at the predetermined level.

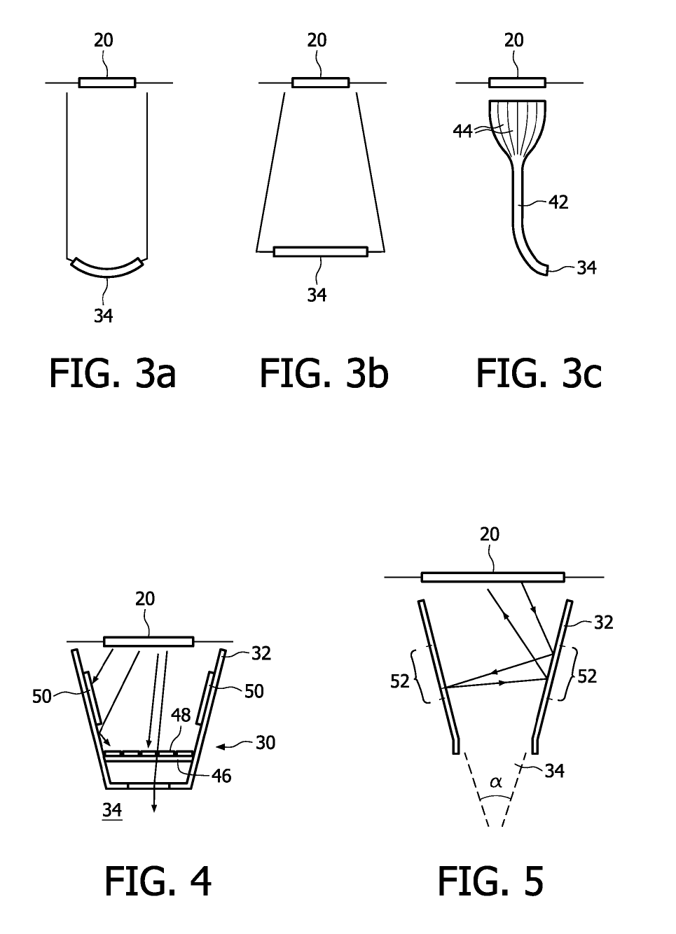

FIGS. 3a-c show some embodiments of adjustment members 30, in a cross-sectional view. In each case, the same first optical window 20 is shown for reference.

FIG. 3a shows an adjustment member 30 with a second optical window 34 that has substantially the same area as window 20 but has a different shape. This shape may be required for e.g. a different treatment, but in particular for the same treatment at a particular or ‘difficult’ spot on the body. Still, without special measures, optical parameter values could change at the second optical window 34, which change is counteracted by the measures according to the invention.

FIG. 3b shows an example of an adjustment member with a second optical window 34 that has a larger area than first window 20. This will always require adaptation of the optical parameters, since otherwise a lower amount of energy would have to be distributed over a larger area. This adjustment member calls for an active control of the light source.

FIG. 3c shows an adjustment member in the form of a lightguide 42 that is composed of a number of optical fibers 44. The fibers 44 are thinner towards their ends that form the second optical window 34. Although, through total internal reflection, the transport is almost lossless, and the properties of the radiation that enters the lightguide 42 are almost the same as those of the light that leaves it at window 34, there will be some absorption, but most importantly, the coupling of the light emitted at the first optical window 20 into the lightguide 42 will not be 100%. Hence, in most cases a correction is required, which may be achieved by selecting a desired diameter change when going from first window 20 to second optical window 34.

FIG. 4 shows another adjustment member according to the invention, in a cross-sectional view.

The adjustment member 30 has a wall 32 with a first coating 50, a second optical window 34, and a carrier 46 with a second coating 48.

The second optical window 34 is smaller than the first window 20. Without measures, the radiation could become too strong at the second optical window 34. A part of the radiation is incident on wall 32, and is either reflected, or absorbed by first coating 50. Similarly, a part of the radiation that is, or would be, incident on the second optical window 34 is absorbed or reflected by first coating 48. In all, the desired amount of radiation is present at the second optical window 34. Another option would be for coating 50 to be diffusely reflecting.

In order to calibrate the adjustment member 30, the width and/or absorption coefficient of first coating 50 and or the area and/or absorption coefficient and/or reflection coefficient of second coating 48 may be suitably selected, if desired, so as to be wavelength-dependent. Moreover, the carrier 46 could also have suitable filter or other absorption properties.

In the case shown, the second optical window 34 could be a protection glass, with hardly any influence on the radiation, but serving to protect the inner parts of the adjustment member 30 and to protect the user against hot filters/carriers 46 et cetera. If the carrier and/or the second optical window 34 is made of untreated glass or the like, this will also show a reflection loss of about 4% at each boundary, depending on the refractive index. If a higher reflection loss would be sufficient to achieve the desired optical parameter value, one or more additional plates of glass or the like could be added.

FIG. 5 shows another embodiment of an adjustment member 30 in cross-sectional view. Herein, a part of the radiation from first optical window 20 is incident on the wall 32 that has a specularly reflecting part 52. A part of the radiation will, after a number of reflections, that depends on the incidence angle and the apical angle α, return to the first optical window, and thus be removed from the radiation through the second optical window 34. The dimensions of the part 52, and the apical angle α determine the ratio, and thus the corresponding optical parameter value.

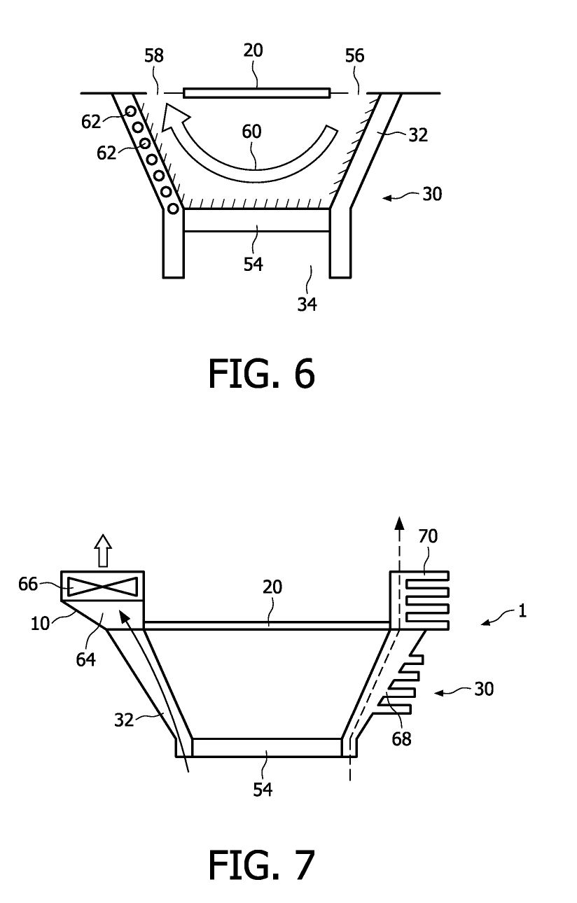

FIG. 6 shows an adjustment member with cooling means, in a cross-sectional view.

Adjustment member 30 with wall 32 is attached in front of and around first optical window 20 and is closed off with a filter 54, a short distance above true second optical window 34. Indicated with a dashed surface are the parts that tend to get hottest when the adjustment member is in use.

Via supply opening 56 and discharge opening 58, a cooling air flow 60 is supplied via pump or fan means (not shown). The air carries off generated heat. Air or other gases are preferable for cooling filters that are hardly in thermal contact with the wall 32.

The adjustment member 30 also comprises a number of coolant channels 62 in the wall 32. Coolant may be supplied to the channel(s) 62 to carry off generated heat. The coolant may be a gas such as air, a fluid such as water, or a mixture. The number and dimensions of the channel(s), the type and flow of the coolant may be selected according to the amount of heat to be carried off. Especially if it is mainly the wall that generates heat, such an internal cooling system is preferable.

FIG. 7 shows (a part of) a system with a number of different cooling means, in a cross-sectional view.

Reference numeral 1 shows a part of a main device, while 30 indicates, as usual, an attached adjustment member.

64 indicates a hollow wall part of the housing 10, with a fan 66. 68 and 70 are a first and second heat sink.

In this case, the wall 32 is also hollow, and connected to the hollow wall part 64. The fan 66 sucks away heated air, or could blow fresh air through part 64 and wall 32. One could also say that the wall 32 has one large channel inside, cfr. channels 62 in FIG. 6.

In the right part of FIG. 7, wall 32 could be hollow, with a similar hollow part of the wall of housing 10 connected thereto, and with a similar, but not-shown fan or the like, to create a coolant flow. This is indicated with a dashed line. The walls could also be solid, and e.g. made of copper or some other material with a high thermal conductivity. To enhance the heat removing properties, first and/or second heat sinks 68, 70 are provided having ribs or other structures to increase their surface area.

1. An optical treatment system, comprising
a housing (10) with a first optical window (20),
a light source (14) in the housing (10) for providing optical radiation,
a control unit (18) for controlling the light source (14) such that the system is able to provide optical radiation at the first optical window (20) with at least one optical parameter having a predetermined first value, and
a set of at least one optical radiation-transmitting adjustment member (30) and a second optical window (34), the adjustment member (30) being positionable with respect to the light source (14) such that at least a part of the optical radiation is emittable via the second optical window (34),
characterized in that the system further comprises an optical parameter control means (36, 38; 42; 46, 48, 50; 52; 54) that is arranged to control said at least one optical parameter to have a predetermined second value at the second optical window (34).

2. An optical treatment system as claimed in claim 1, wherein the optical parameter control means (36, 38; 42; 46, 48, 50; 52; 54) is arranged to control said optical parameter in dependence on at least one of a shape and a dimension of said second optical window (34) and/or adjustment member (30).

3. An optical treatment system as claimed in claim 1, wherein the optical parameter comprises at least one of a spectrum, an intensity and a fluence of the optical radiation, and preferably the intensity.

4. An optical treatment system as claimed in claim 1, wherein the second value is substantially equal to the first value.

5. An optical treatment system as claimed in claim 1, wherein the optical parameter control means comprises an identification means (36, 38) that is able to co-operate with the control unit (18), or a detector means (38) arranged to detect the presence of the adjustment member (30) and preferably to detect a type of the adjustment member (30).

6. An optical treatment system as claimed in claim 1, wherein the optical parameter control means comprise a passive radiation-influencing means (46, 48, 50; 52; 54), that is arranged to manipulate the optical radiation emitted by the light source (14) or to manipulate the optical radiation emitted through the first optical window (20).

7. An optical treatment system as claimed in claim 1, wherein the adjustment member (30) comprises at least one optical element (34; 46, 48; 54).

8. An optical treatment system as claimed in claim 7, wherein the optical element comprises a filter (48; 54), or a carrier (46) with a coating (48) that is partly reflective and/or absorptive.

9. An optical treatment system as claimed in claim 1, wherein the adjustment member (30) comprises at least one lightguide (42), preferably a bundle of optical fibers (44), with a diameter that decreases towards the second optical window (34).

10. An optical treatment system as claimed in claim 1, wherein the system comprises cooling means (60, 62; 64, 66, 68, 70) arranged to remove heat from the adjustment member (30).

11. An optical treatment system as claimed in claim 1, wherein the cooling means comprise a coolant fluid channel (62; 64) in the adjustment member (30) for flowing coolant fluid, or a gas flow (60) in an inner space of the adjustment member (30).

12. An adjustment member (30) for use in the optical treatment system according to claim 1.
